(19) United States
(12) Patent Application Publication
(10) Pub. No.: US 2026/0315559 A1
(43) Pub. Date: Oct. 15, 2026

(54) INJECTABLE COMPOSITION FOR TREATMENT OF CHRONIC OBESITY

(71) Applicant: MyoungSeok CHOI, Seoul (KR)

(72) Inventor: MyoungSeok CHOI, Seoul (KR)

(21) Appl. No.: 19/178,750

(22) Filed: Apr. 14, 2025

(30) Foreign Application Priority Data
Apr. 10, 2025 (KR) 10-2025-0046879

(51) Int. Cl. A61K 36/16; A61K 9/00; A61K 31/167; A61K 31/375; A61K 31/522; A61K 36/48; A61K 47/02; A61P 3/04

(52) U.S. Cl. CPC A61K 36/16; A61K 9/0019; A61K 31/167; A61K 31/375; A61K 31/522; A61K 36/48; A61K 47/02; A61P 3/04

(57) ABSTRACT

The object of the present invention is to provide an injectable composition for treating chronic obesity, which can decompose accumulated fat that is not easily removed from or lost in a chronically obese patient by general methods, while minimizing damage to the liver, heart, kidneys, etc. To this end, the injectable composition for treating chronic obesity of the present invention is characterized in that it comprises, as active ingredients, 0.15 to 0.2 wt % of pentoxifylline, 0.4 to 0.5 wt % of aminophylline, 0.3 to 0.4 wt % of a melilotus extract, 0.05 to 0.07 wt % of a Ginko biloba extract, and the remainder of normal saline, based on the weight ratio of the total composition, and is administered directly into subcutaneous fat tissue at

[symbol]

### CROSS-REFERENCE TO RELATED APPLICATION

This application claims the benefit of Korean Patent Application No. 10-2025-0046879 filed on Apr. 10, 2025, in the Korean Intellectual Property Office, the entire disclosure of which is incorporated herein by reference for all purposes.

### BACKGROUND OF THE INVENTION

The present invention relates to an injectable composition for treatment of chronic obesity.

It is generally recommended to reduce calorie intake and increase calorie consumption through methods such as exercise in order to treat obesity. Surgical methods such as liposuction and gastric resection surgery are also used to reduce excessive fat accumulation.

Methods for reducing calorie intake comprise reducing calorie intake by one's own will, using drugs such as appetite suppressants, and using surgical methods such as gastrectomy to reduce stomach volume. Medical methods for reducing excessive subcutaneous fat comprise methods such as lipolysis using drugs that act on osmotic pressure and fat cells, methods using drugs such as lipolysis injections, physical methods for reducing subcutaneous fat using high frequency, laser, ultrasound, etc., and liposuction and lipectomy for surgically removing fat.

If such calorie restriction and increased calorie expenditure are combined with additional surgical treatment, theoretically anyone should be able to achieve the slim figure they want.

However, in reality, especially in cases of severe obesity where chronic, long-term accumulated fat is the problem, only a very small number of cases achieve desired results even if the above-described three methods are used simultaneously.

In other words, unlike sudden weight gain, chronic long-term obesity patients show abnormalities in fat metabolism and low-level chronic inflammation in the area where old fat is accumulated, and the accumulated fat in that area is not easily used as an energy source, and abnormalities in lymphatic circulation are pointed out as the cause of these changes. Therefore, if chronic lymphatic and blood circulation disorders in these areas of old fat accumulation are treated, the inflammatory state of the fat tissue and abnormalities in fat metabolism will also be treated, and, as a result, the accumulated fat with old ischemia and inflammation that was not easily used as an energy source may be used as an energy source, allowing escape from the vicious cycle of obesity. In modern medicine, physical therapy using high frequency, ultrasound, laser, and electrical stimulation is used to treat these areas, but the effects are minimal. As an alternative, drugs that affect lymphatic and blood circulation may be taken orally or injected into the blood and muscles. However, drugs taken orally are absorbed through the digestive system, delivered to the heart through the liver, and then spread throughout the body. Injected drugs are absorbed into the venous blood through muscles with a large blood distribution in the case of intramuscular injection, and are directly absorbed into the venous blood in the case of intravascular injection and reach the target area through the portal vein, heart, and arteries. Therefore, in order for a significant amount of drugs to reach the actual target area, the old subcutaneous fat layer, a considerable amount of drugs must be taken or injected. In this case, there is a problem that the high concentration of drugs increases the possibility of damage to the liver, heart, and kidneys, where the drugs are excreted.

### SUMMARY OF THE INVENTION

The object of the present invention is to provide an injectable composition for treating chronic obesity, which may decompose accumulated fat that is not easily removed from or lost in chronic, long-term obese patients by general methods, while minimizing damage to the liver, heart, kidneys, etc.

In order to achieve the above-described object of the present invention, the present invention provides an injectable composition for treating chronic obesity according to the embodiment of the present invention, which is characterized in that the composition comprises, as active ingredients, 0.15 to 0.2 wt % of pentoxifylline, 0.4 to 0.5 wt % of aminophylline, 0.3 to 0.4 wt % of a melilotus extract, 0.05 to 0.07 wt % of a Ginko biloba extract, and the remainder of normal saline, based on the weight ratio of the entire composition, and the composition is administered directly into subcutaneous fat tissue at checkerboard-shaped intervals.

The above-described injectable composition for treating chronic obesity according to the embodiment of the present invention may further comprise, as an active ingredient, 0.3 to 0.4 wt % of vitamin C, based on the weight ratio of the entire composition.

The above-described injectable composition for treating chronic obesity according to the embodiment of the present invention may further comprise, as an active ingredient, 0.07 to 0.08 wt % of lidocaine, based on the weight ratio of the entire composition.

The above-described injectable composition for treating chronic obesity according to the embodiment of the present invention may comprise active ingredients consisting of 50 mg of the pentoxifylline, 125 mg of the aminophylline, 100 mg of the melilotus extract, 17.5 mg of the Ginko biloba extract, 100 mg of the vitamin C, 20 mg of the lidocaine, and the remainder of the normal saline.

When the above-described injectable composition for treating chronic obesity according to the embodiment of the present invention is administered as a single injection dose to the subcutaneous fat tissue of the whole body of a chronic obese patient, the single injection dose is 25 to 30 cc, and the single injection dose may be administered to multiple points in the subcutaneous fat tissue of the whole body of the chronic obese patient at checkerboard-shaped intervals.

The single injection dose is divided and administered to each of the points, and a relatively larger amount may be administered to a point with a relatively high degree of obesity among the multiple points.

As described above, the injectable composition for treating chronic obesity according to the embodiment of the present invention can have the following effects.

According to the embodiment of the present invention, there are provided technical features in that the injectable composition for treating chronic obesity comprises 0.15 to 0.2 wt % of pentoxifylline, 0.4 to 0.5 wt % of aminophylline, 0.3 to 0.4 wt % of a melilotus extract, 0.05 to 0.07 wt % of a Ginko biloba extract, and the remainder of normal saline, based on the weight ratio of the entire composition, and is administered directly into subcutaneous fat tissue at checkerboard-shaped intervals. It was confirmed in an experiment targeting patients that the composition having such weight ratios of components can significantly decompose the old fat tissue of chronic obese patients and maintain the concentration of the effective drug in the old fat tissue of chronic obese patients higher than compositions taken orally or injected into blood vessels or muscles, without using a large amount of drugs by direct administration to the subcutaneous fat tissue. In other words, when the composition is directly administered to the old subcutaneous fat layer where the tissue is degenerated, the highest drug concentration can be maintained in the fat layer, which is the target area, such that damage to the liver, heart, and kidneys can be minimized. In the case of blood injection or muscle injection, the highest drug concentration is shown in the injected vein or muscle, but the concentration of the drug that passes through each organ and actually reaches the subcutaneous fat layer is extremely small. Thus, in order to increase the drug concentration in the subcutaneous fat layer by blood and muscle injection and oral administration, a very large amount of drug must be administered, and, in such case, drug toxicity and side effects increase, thereby causing damage to the liver, heart, and kidneys.

In addition, according to the embodiment of the present invention, there is provided a technical feature in that the composition further comprises, as an active ingredient, 0.3 to 0.4 wt % of vitamin C, based on the weight ratio of the entire composition, thereby increasing collagen synthesis to provide elasticity to skin sagging due to fat decomposition.

In addition, according to the embodiment of the present invention, there is provided a technical feature in that the composition further comprises, as an active ingredient, 0.07 to 0.08 wt % of lidocaine, based on the weight ratio of the entire composition, thereby preventing pain caused by drug stimulation after injection.

In addition, according to the embodiment of the present invention, there is provided a technical feature in that, when the composition is administered to the whole body in a single injection dose, the single injection dose is 25 to 30 cc, and the single injection dose is administered to multiple points in the subcutaneous fat tissue of the whole body at checkerboard-shaped intervals. Therefore, the single injection dose can be divided and administered to the whole body, such that even a small amount of the drug can effectively decompose fat in the subcutaneous fat tissue of the whole body.

In addition, according to the embodiment of the present invention, there is provided a technical feature in that a single injection dose is divided and administered to each of the points, and a relatively large amount is administered to a point with a relatively high degree of obesity among the multiple points, such that fat can be broken down more quickly at a point with a high degree of obesity to increase patient satisfaction with whole-body obesity treatment.

### BRIEF DESCRIPTION OF THE DRAWINGS

FIG. 1 shows the change in the body indices of each of ten (10) test group subjects administered with the injectable composition for treating chronic obesity according to the embodiment of the present invention.

FIG. 2 shows the change in the body indices of each of ten (10) control group subjects administered with the injectable composition of the comparative example.

### DETAILED DESCRIPTION OF THE INVENTION

Hereinafter, with reference to the attached drawings, embodiments of the present invention will be described in detail so that those skilled in the art can easily implement the present invention. However, the present invention may be implemented in various different forms and is not limited to the embodiments described herein.

An injectable composition for treating chronic obesity according to the embodiment of the present invention comprises, as active ingredients, pentoxifylline, aminophylline, melilotus extract, Ginko biloba extract, and normal saline.

The present invention is characterized by a novel injectable composition comprising, as active ingredients, pentoxifylline, aminophylline, melilotus extract, Ginko biloba extract, and normal saline, and being administered directly into the subcutaneous fat tissue of a chronically obese patient at checkerboard-shaped intervals by using a needle to treat chronic obesity.

As used herein, the “Pentoxifylline” is a competitive non-selective phosphodiesterase inhibitor that, like other methylated xanthine derivatives, increases intracellular CAMP, activates PKA, inhibits TNF and leukotriene synthesis, and reduces inflammation and innate immunity. In addition, pentoxifylline improves red blood cell deformability (called hematological effect), lowers blood viscosity, and reduces platelet aggregation and thrombus formation potential. Pentoxifylline is also an antagonist of the adenosine 2 receptor.

Also, pentoxifylline, like aminophylline, is a drug belonging to the xanthine (xanthine) derivatives that increases energy levels by increasing intracellular CAMP, and tumor necrosis factor-α (TNF-α) is a cytokine that is involved in the inflammatory response and is a member of the acute-phase protein. TNF-α is mainly secreted by activated macrophages, and is also secreted by various cells such as helper T cells, natural killer cells, and damaged neurons. The most important role of TNF-α is the regulation of immune cells. TNF-α is a pyrogen in the body and has the ability to induce fever, induce apoptosis, induce sepsis through the production of IL-1 and IL-6, induce cachexia, induce infection, and inhibit tumorigenesis and viral replication. Abnormal regulation of TNF-α has been shown in various human diseases such as Alzheimer's disease, cancer, depression, and inflammatory bowel disease (IBD).

In particular, TNF-α has the effect of blocking the mechanism of chronic obesity by improving the inflammatory state caused by chronic obesity by suppressing the inflammatory response through the reduction of TNF tumor necrosis factor, and also has the effect of promoting blood circulation by reducing blood viscosity and inhibiting thrombosis.

In addition, in the injectable composition for treating chronic obesity according to the embodiment of the present invention, pentoxifylline is preferably blended in an appropriate amount in the range of 0.15 to 0.2 wt %, based on the weight ratio of the entire composition. When the amount was less than 0.15 wt %, the composition had no effective effect on suppressing inflammation response, and, when the amount exceeded 0.2 wt %, the composition showed a minimal synergistic effect on suppressing inflammation response.

In addition, as used herein, the “aminophylline” is a xanthine drug that exhibits a lipolytic effect with an action similar to adrenaline.

In addition, in the injectable composition for treating chronic obesity according to the embodiment of the present invention, aminophylline is preferably blended in an appropriate amount in the range of 0.4 to 0.5 wt %, based on the weight ratio of the entire composition. When the amount was less than 0.4 wt %, the composition had no effective effect on lipolysis, and, when the amount exceeded 0.5 wt %, the composition showed a minimal synergistic effect on lipolysis.

In addition, as used herein, the “melilotus extract” prevents lymphedema, venous insufficiency, hemorrhoids, varicose veins, perineal incision, post-traumatic inflammation, filaritic lymphedema and epithelial disease, cancer (e.g., malignant melanoma, renal cell carcinoma, prostate carcinoma), especially their metastases. By improving lymphatic function, melilotus increases venous return and is beneficial for post-operative edema.

In particular, the extract of melilotus is a drug used for these diseases and has the effects of promoting lymphatic circulation, promoting blood circulation, reducing edema, and suppressing inflammation, thereby normalizing the chronic lymphatic circulation abnormality and inflammation state of chronic obese tissue, and the chronic ischemic state of blood circulation deficiency.

In addition, in the injectable composition for treating chronic obesity according to the embodiment of the present invention, the melilotus extract is preferably blended in an appropriate amount in the range of 0.3 to 0.4 wt %, based on the weight ratio of the entire composition. When the amount is less than 0.3 wt %, the composition had no effective effect on promoting lymphatic circulation and blood circulation, reducing edema, and inhibiting inflammation, and, when the amount exceeded 0.4 wt %, the composition showed a minimal synergistic effect on promoting lymphatic circulation and blood circulation, reducing edema, and inhibiting inflammation.

In addition, as used herein, the “Ginko biloba extract” has long been used to treat blood diseases and memory problems. Today, using same is best known as a way to keep memory sharp. Laboratory studies have shown that ginkgo improves blood circulation by dilating blood vessels and making blood less sticky. It is also an antioxidant.

In particular, Ginko biloba extract is effective in normalizing chronic obesity tissue through its anti-inflammatory action and promotion of lymphatic drainage.

In addition, in the injectable composition for treating chronic obesity according to the embodiment of the present invention, the melilotus extract is preferably blended in an appropriate amount in the range of 0.05 to 0.07 wt %, based on the weight ratio of the entire composition. When the amount is less than 0.05 wt %, the composition had no effective effect on anti-inflammatory action and promotion of lymphatic drainage, and, when the amount exceeded 0.07 wt %, the composition showed a minimal synergistic effect on anti-inflammatory action and promotion of lymphatic drainage.

In addition, as used herein, the “normal saline” has the purpose of acting as a buffer for the mixed drug and controlling the drug volume. Such normal saline forms the remainder, based on the weight ratio of the entire composition.

In addition, the injectable composition for treating chronic obesity according to the embodiment of the present invention may further comprise vitamin C as an active ingredient. As used herein, the “vitamin C” has the effect of promoting the recovery of skin elasticity after subcutaneous fat is reduced by increasing collagen synthesis.

In addition, in the injectable composition for treating chronic obesity according to the embodiment of the present invention, vitamin C is preferably blended in an appropriate amount in the range of 0.3 to 0.4 wt %, based on the weight ratio of the entire composition. When the amount was less than 0.3 wt %, the composition had no effective effect on increasing collagen synthesis, and, when the amount exceeded 0.4 wt %, the composition showed a minimal synergistic effect on increasing collagen synthesis and induced irritation symptoms (e.g., pain and redness) at the injection site.

In addition, the injectable composition for treating chronic obesity according to the embodiment of the present invention may further comprise lidocaine as an active ingredient. As used herein, the “lidocaine” has the effect of preventing pain caused by drug stimulation after injection.

In addition, in the injectable composition for treating chronic obesity according to the embodiment of the present invention, lidocaine is preferably blended in an appropriate amount in the range of 0.07 to 0.08 wt %, based on the weight ratio of the entire composition. When the amount is less than 0.07 wt %, the composition had no effective effect on pain prevention, and, when the amount exceeded 0.08 wt %, the composition had a minimal synergistic effect on pain prevention and rarely induced side effects such as myocardial contractility abnormalities, arrhythmia, and convulsions.

As described above, in order to maximize the obesity treatment of the injectable composition for treating chronic obesity according to the embodiment of the present invention, the amount ratio of pentoxifylline, aminophylline, melilotus extract, Ginko biloba extract, vitamin C, lidocaine, and normal saline should be optimized. Preferably, as described above, the injectable composition for treating chronic obesity according to the embodiment of the present invention comprises 0.15 to 0.2 wt % of pentoxifylline, 0.4 to 0.5 wt % of aminophylline, 0.3 to 0.4 wt % of melilotus extract, 0.05 to 0.07 wt % of Ginko biloba extract, 0.3 to 0.4 wt % of vitamin C, 0.07 to 0.08 wt % of lidocaine C, and 98.4 to 98.5 wt % of normal saline.

Most preferably, the injectable composition for treating chronic obesity according to the embodiment of the present invention may comprise active ingredients consisting of 50 mg of pentoxifylline, 125 mg of aminophylline, 100 mg of melilotus extract, 17.5 mg of Ginko biloba extract, 100 mg of vitamin C, 20 mg of lidocaine, and the remainder being normal saline.

With such active ingredients, the injectable composition for treating chronic obesity according to the embodiment of the present invention, when administered to the whole body of a chronic obese patient as a single injection dose, is administered at a single injection dose of 25 to 30 cc (most preferably, 27 cc), and may be administered as a single injection dose to multiple points in the subcutaneous fat tissue of the whole body of the chronic obese patient at checkerboard-shaped intervals. For example, when a single injection dose is administered to 200 places (points) of the whole body, the approximate gap of points to be administered may be determined by dividing the surface area of the whole body of the chronic obese patient by 200. In particular, the single injection dose is divided and administered to each of the points, and a relatively large amount may be administered to a point with a relatively high degree of obesity among the multiple points.

Continuing below, an experimental example using an injectable composition for treating chronic obesity prepared through the embodiment of the present invention is described.

### Experimental Example

An injectable composition was provided with the composition shown in Table 1 below.

TABLE 1
Injectable composition

| Ingredient | Comparative Example | Present invention | |
|---|---|---|---|
| Pentoxifylline | — | 50 | mg |
| Aminophylline | — | 125 | mg |
| Melilotus extract | — | 100 | mg |
| Ginko biloba extract | — | 17.5 | mg |
| Vitamin C | — | 100 | mg |
| Lidocaine | — | 20 | mg |

| | | |
|---|---|---|
| Normal saline | Remaining amount | Remaining amount |
| Total amount (cc) | 27 | 27 |

### Human Clinical Trials for Obesity Treatment Effects

The obesity treatment effect of the injectable composition for treating chronic obesity according to the embodiment of the present invention was confirmed through a clinical trial. The subjects of the experiment were adults with chronic obesity aged 19 to 60 years, and volunteers were recruited publicly.

A total of 20 applicants were divided into two groups of 10 each, and one group (test group) was administered with the injectable composition of the present invention as shown in Table 1, and the other group (control group) was administered with the injectable composition of the comparative example. Both groups were administered with the same amount of the composition at 1-week intervals for 24 weeks (6 months). In addition, the injectable composition was administered using an automatic digital painless syringe injector, enabling painless quantitative treatment.

The results of comparing the changes in body indices before and after administration of the injectable composition of the present invention are as shown in Table 2 below. In the test group, there was a decrease in all body indices comprising weight and body fat. On the other hand, in the case of the control group, the recipients were administered without knowing that they were in the control group, and were administered with an injectable composition containing only normal saline. Although there was a slight weight loss due to the placebo effect, it was found that the weight loss was significantly less than that of those administered with the injectable composition of the present invention.

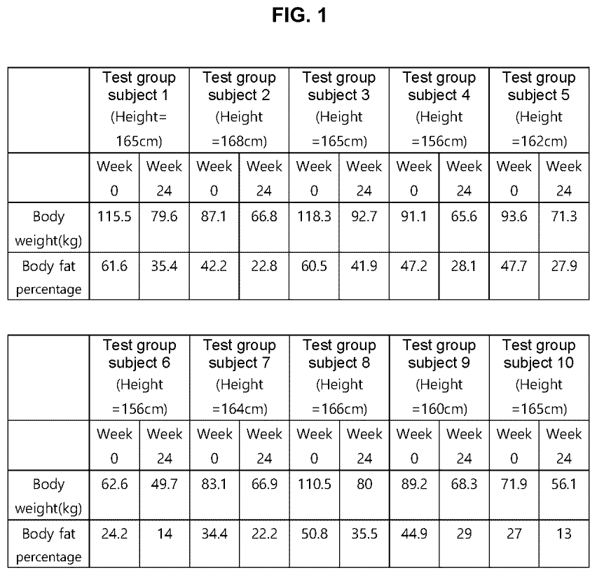

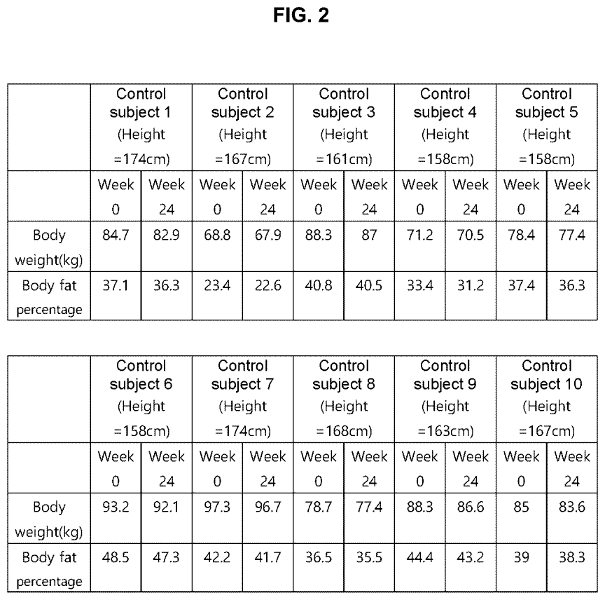

For reference, the size and body fat mass are the results measured using the InBody body composition analyzer, and FIG. 1 shows the change in body indices of each of the ten test group members, and FIG. 2 shows the change in body indices of each of the ten control group members.

TABLE 2
Changes in body indices before and after
administration (average of 10 subjects)

| | Group A (test group) | | Group B (control group) | |
|---|---|---|---|---|
| | (n = 10) | | (n = 10) | |
| | Week 0 | Week 24 | Week 0 | Week 24 |
| Body weight (kg) | 91.29 | 69.70 | 83.39 | 82.21 |
| Body fat | 44.05 | 26.98 | 38.27 | 37.29 |
| percentage (%) | | | | |

As shown in Table 2, it was confirmed that the obesity treatment effect in chronically obese patients was superior in the test group administered with the injectable composition of the present invention, as compared to the control group administered with the injectable composition of the comparative example.

### Evaluation of Side Effects of the Injectable Composition of the Present Invention

The side effects observed in each patient by administering the injectable composition of the present invention through clinical trials are shown in Table 3 below.

TABLE 3

| | | | | | Skin | Sagging | Skin |
|---|---|---|---|---|---|---|---|
| | Redness | swelling | itching | Pain | depression | skin | elasticity |
| Present | 1 | 0 | 0 | 0 | 0 | 0 | 5 |
| invention | | | | | | | |

In Table 3, the item “skin elasticity” was measured on a 5-point scale by touch, and was categorized as 1: very bad, 2: bad, 3: average, 4: slightly good, and 5: good. The remaining items in Table 3 indicate the number of people.

It was found that the injectable composition of the present invention showed significantly reduced allergic redness, swelling, and itching, and, by containing lidocaine, the composition had no pain (pain of VAS 3 or higher), and, by containing vitamin C, the composition showed quickly restored skin elasticity, without the occurrence of skin depression or sagging skin.

In a comprehensive review, a small amount of the drug evenly decomposed fat throughout the body, caused no pain, and did not cause skin depression or sagging skin due to fat decomposition, thereby maintaining skin elasticity.

Although the preferred embodiments of the present invention have been described in detail above, the scope of the present invention is not limited thereto. In addition, various modifications and improvements made by those skilled in the art using the basic concept of the present invention defined in the following claims also fall within the scope of the present invention.

What is claimed:

1. An injectable composition for treating chronic obesity, comprising, as active ingredients, 0.15 to 0.2 wt % of pentoxifylline, 0.4 to 0.5 wt % of aminophylline, 0.3 to 0.4 wt % of a melilotus extract, 0.05 to 0.07 wt % of a Ginko biloba extract, and the remainder of normal saline, based on the weight ratio of the entire composition,
wherein the composition is administered directly into subcutaneous fat tissue at checkerboard-shaped intervals.

2. The injectable composition for treating chronic obesity of claim 1,
wherein the composition further comprises, as an active ingredient, 0.3 to 0.4 wt % of vitamin C, based on the weight ratio of the entire composition.

3. The injectable composition for treating chronic obesity of claim 2,
wherein the composition further comprises, as an active ingredient, 0.07 to 0.08 wt % of lidocaine, based on the weight ratio of the entire composition.

4. The injectable composition for treating chronic obesity of claim 3,
wherein the active ingredient consists of 50 mg of the pentoxifylline, 125 mg of the aminophylline, 100 mg of the melilotus extract, 17.5 mg of the Ginko biloba extract, 100 mg of the vitamin C, 20 mg of the lidocaine, and the remainder of the normal saline.

5. The injectable composition for treating chronic obesity of claim 4,
wherein, when the composition is administered to the whole body as a single injection dose, the single injection dose is 25 to 30 cc, and the single injection dose is administered to multiple points in the subcutaneous fat tissue of the whole body at checkerboard-shaped intervals.

6. The injectable composition for treating chronic obesity of claim 5,
wherein the composition is administered dividedly to each of the points, and a relatively large amount is administered to a point with a relatively high degree of obesity among the multiple points.
